(19) United States
(12) Patent Application Publication
(10) Pub. No.: US 2023/0197238 A1
(43) Pub. Date: Jun. 22, 2023

(54) DEVICE FOR ASSISTING WITH IMPROVEMENT IN ACTIVITIES OF DAILY LIVING

(71) Applicant: CARE SMILE AOMORI CO., LTD, Aomori-shi, Aomori (JP)

(72) Inventor: Hiroshi OSATO, Aomori (JP)

(21) Appl. No.: 17/995,946

(22) PCT Filed: Oct. 29, 2021
(86) PCT No.: PCT/JP2021/039964

§ 371 Date: Oct. 11, 2022

(30) Foreign Application Priority Data
Dec. 25, 2020 (JP) 2020-217327

(51) Int. Cl. G16H 20/30; G09B 19/00

(52) U.S. Cl. CPC G16H 20/30; G09B 19/003

(57) ABSTRACT

To enable improvement in the activities of daily living. A device for assisting with improvement in ADLs stores in advance training indicators defining four types of movements associated with at least four stages of human developmental movements and their sequence, reference images of the four types of movements, and reference scores for evaluating a training subject's level of self-reliance for each training indicator in a storage unit; processes a first training program adopting a basic sequence of the four types of movements for display on a display unit, upon being triggered by the start of initial training; screens for acquired movements based on the first training program to determine an achievement score for the training subject, and for each training indicator, processes the achievement score for display and storages in the storage unit; processes a second training program adopting the basic sequence or a modified sequence of the four types of movements according to the achievement score for each training indicator for display on the display unit, upon being triggered by the start of ongoing training; and screens for acquired movements based on the second training program to determine an achievement score for the training subject, and for each training indicator, processes the achievement score for display.

### TECHNICAL FIELD

The present invention relates to a device for assisting with improvement in the activities of daily living, and furthermore relates to a method for assisting with improvement in the activities of daily living and a program for assisting with improvement in the activities of daily living.

### BACKGROUND ART

Recently, as the demographic composition becomes an inverted pyramid and heads toward a superannuated society, there is a need for technologies for assisting with improvement in the activities of daily living that help training subjects, including persons requiring assistance, improve their activities of daily living.

Examples of such assisting technologies are proposed in Patent Literature 1, 2, 3, and 4, for example.

### RELATED ART LITERATURE

### Patent Literature

Patent Literature 1: Japanese Patent Laid-Open No. 2018-013967

Patent Literature 2: Japanese Patent Laid-Open No. 2017-027192

Patent Literature 3: Japanese Patent Laid-Open No. 2015-148915

Patent Literature 4: Japanese Patent Laid-Open No. 2011-013834

### SUMMARY OF INVENTION

### Problems to be Solved by the Invention

However, Patent Literature 1, 2, 3, and 4 cited above do not disclose a technology for assisting with improvement in the activities of daily living that rapidly screens for movements that a training subject needs to acquire at the present time and prescribes a proper training regimen for the movements that need to be acquired.

The objective is to provide a technology for assisting with improvement in the activities of daily living that rapidly screens for movements that a training subject needs to acquire at the present time and prescribes a proper training regimen for the movements that need to be acquired.

### Means for Solving the Problems

To address the above problem, a device for assisting with improvement in activities of daily living according to one aspect provides a training program for assisting with improvement in the activities of daily living for a training subject, the device comprising a processor configured to: store in advance training indicators defining four types of movements associated with at least four stages of human developmental movements and their sequence, reference images of the four types of movements, and reference scores for evaluating a training subject's level of self-reliance for each training indicator in a storage unit; process a first training program adopting a basic sequence of the four types of movements for display on a display unit, upon being triggered by the start of initial training for the training subject; screen for acquired movements based on the first training program to determine an achievement score for the training subject, and for each training indicator, process the achievement score for display on the display unit and store the achievement score in the storage unit; process a second training program adopting the basic sequence or a modified sequence of the four types of movements according to the achievement score for each training indicator for display on the display unit, upon being triggered by the start of ongoing training for the training subject; and screen for acquired movements based on the second training program to determine an achievement score for the training subject, and for each training indicator, process the achievement score for display on the display unit and store the achievement score in the storage unit.

In the above aspect, the four stages of developmental movements and their sequence correspond to original fundamental human movement patterns, and include abdominal pressure and pelvic stability, trunk stability, center of gravity shift and bipedal support, and left-right asymmetry and single leg support.

In the above aspect, the four types of movements are movements fundamental to the activities of daily living, and include straight leg raising, seated shoulder raising, standing up from a chair, and standing on one leg as first, second, third, and fourth training indicators, respectively.

In the above aspect, the basic sequence of the four types of movements is the sequence of the first training indicator, the second training indicator, the third training indicator, and the fourth training indicator.

In the above aspect, the modified sequence of the four types of movements is a sequence omitting at least one subordinate indicator of the first training indicator, the second training indicator, the third training indicator, and the fourth training indicator.

In the above aspect, the first training indicator, the second training indicator, the third training indicator, and the fourth training indicator of the four types of movements each include three sub-movements categorized into different difficulties corresponding to a plurality of different scores.

In the above aspect, the first training indicator, the second training indicator, the third training indicator, and the fourth training indicator of the four types of movements each include three sub-movements categorized into different difficulties corresponding to a plurality of different scores, and

the processor is configured to determine the achievement score for the training subject on a basis of a positive or negative determination regarding an acquired movement to be obtained via a selective designation with respect to the three sub-movements categorized into different difficulties.

In the above aspect, the first training indicator, the second training indicator, the third training indicator, and the fourth training indicator of the four types of movements each include three sub-movements categorized into different difficulties corresponding to a plurality of different scores, and

the processor is configured to determine the achievement score for the training subject on a basis of a positive or negative determination regarding an acquired movement to be obtained by image analysis via image capture of the three sub-movements categorized into different difficulties.

In the above aspect, when the achievement score is low for all of the first training indicator, the second training indicator, the third training indicator, and the fourth training indicator of the four types of movements, the processor is configured to generate the second training program in the basic sequence.

In the above aspect, when the achievement score is the same in adjacent sequence of the first training indicator, the second training indicator, the third training indicator, and the fourth training indicator of the four types of movements, the processor is configured to generate the second training program in a modified sequence adopting the lower-order training indicator.

In the above aspect, when the first training indicator, the second training indicator, the third training indicator, and the fourth training indicator of the four types of movements indicate different evaluations on the left and right sides of the training subject's body, the processor is configured to generate the second training program in a modified sequence adopting the training indicator with the lower achievement score.

In the above aspect, the processor is configured to evaluate the training subject's level of self-reliance with consideration for both a score on the Barthel Index which is an index indicating the training subject's level of independence in the activities of daily living, and the achievement score for the training subject in the four types of movements.

In the above aspect, when the index indicating the level of independence in the activities of daily living indicates different evaluations on the left and right sides of the training subject's body, the processor is configured to adopt the higher score.

The features of the device for assisting with improvement in the activities of daily living in each of the aspects described above may also be carried out as a method for assisting with improvement in the activities of daily living, and furthermore may be carried out as a program for assisting with improvement in the activities of daily living that causes a processor to execute the method for assisting with improvement in the activities of daily living.

### Effects of the Invention

According to the disclosed technology, it is possible to rapidly screen for movements that a training subject needs to acquire at the present time and prescribe a proper training regimen for the movements that need to be acquired, thereby leading to improvements in the activities of daily living for the training subject.

Other objects, features and advantages will become apparent from a reading of the description of embodiments for carrying out the invention set forth below when taken in conjunction with the drawings and claims.

### BRIEF DESCRIPTION OF THE DRAWINGS

FIG. 1 is a diagram for explaining functions of a device for assisting with improvement in the activities of daily living according to an embodiment;

FIG. 2 is a diagram for explaining the configuration and functions of a device for assisting with improvement in the activities of daily living according to an embodiment;

FIG. 3 is a diagram for explaining a process for assisting with improvement in the activities of daily living according to an embodiment;

FIG. 4 is a diagram for explaining a process for assisting with improvement in the activities of daily living according to an embodiment;

FIG. 5 is a diagram for explaining a first training indicator in a process according to an embodiment;

FIG. 6 is a diagram for explaining a second training indicator in a process according to an embodiment;

FIG. 7 is a diagram for explaining a third training indicator in a process according to an embodiment;

FIG. 8 is a diagram for explaining a fourth training indicator in a process according to an embodiment;

FIG. 9 is a diagram for explaining a Barthel index in a process according to an embodiment; and

FIG. 10 is a diagram for explaining a process for assisting with improvement in the activities of daily living according to an embodiment.

### MODE FOR CARRYING OUT THE INVENTION

Hereinafter, a more detailed description will be provided with reference to the attached drawings. A preferred embodiment is illustrated in the drawings. However, the invention may also be carried out in many different modes, and is not limited to the embodiment set forth in this specification.

[Device for Assisting with Improvement in the Activities of Daily Living]

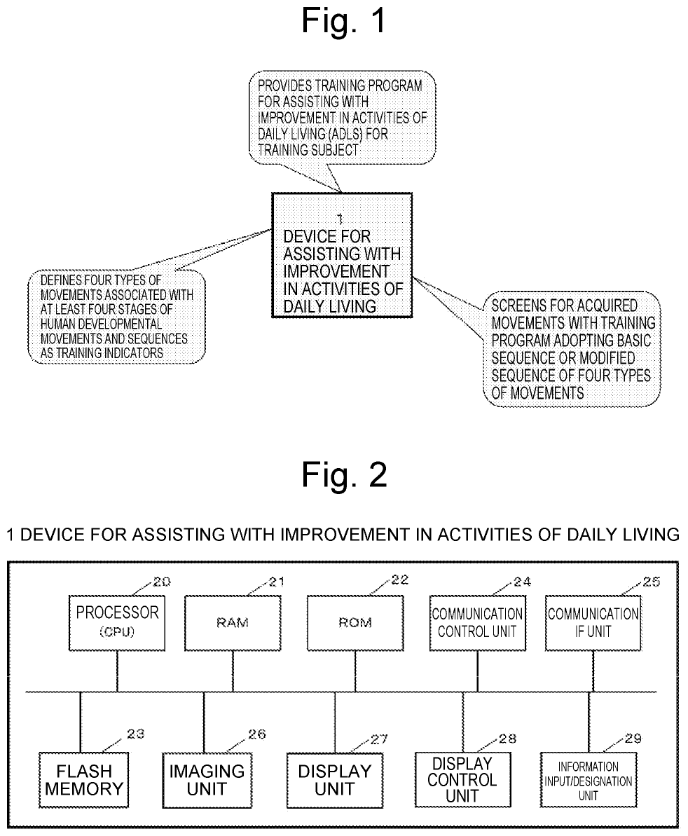

Referring to FIG. 1, a device 1 for assisting with improvement in the activities of daily living (also referred to as the device 1 for assisting with improvement in ADLs) according to an embodiment is installed in a stationary or movable way in a training facility and provides a training program (exercise program) for assisting with improvement in the activities of daily living (ADLs) for a training subject who utilizes the training facility.

By training (giving prescriptions for) many training subjects, including persons requiring assistance and the like, the inventor of the device 1 for assisting with improvement in the ADLs disclosed herein has recognized the need for not only better partial motion but also better movement for improving the activities of daily living (also simply referred to as the ADLs) for training subjects. In other words, achieving better motion of joints and parts of the body is inadequate at achieving better movement for a training subject. This is because achieving better movement further requires motor control abilities such as balance, posture control, cognition, perception, and coordination. Consequently, the inventor focused on the fact that achieving better movement leads to improvements in the activities of daily living.

One important element in the device 1 for assisting with improvement in the ADLs is that four types of movements associated with at least four stages of human developmental movements and their sequence are defined as training indicators. Namely, the four stages of developmental movements and their sequence include abdominal pressure and pelvic stability, trunk stability, center of gravity shift and bipedal support, and left-right asymmetry and single leg support, which correspond to original fundamental human movement patterns.

Additionally, the four types of movements associated with the four stages of developmental movements and their sequence are movements fundamental to the activities of daily living, and include straight leg raising (active-straight leg raising (A-SLR)), seated shoulder raising, standing up from a chair, and standing on one leg as first, second, third, and fourth training indicators, respectively. Strictly speaking, the straight leg raising is active straight leg raising, and the standing on one leg is standing on one leg with eyes open. Also, the seated shoulder raising may also be referred to as seated shoulder flexion.

In the device 1 for assisting with improvement in the ADLs, a training subject is screened for acquired movements according to a training program which is based on original fundamental human movement patterns and in which a basic sequence or a modified sequence of the four types of movements corresponding to the movement patterns is adopted.

In FIG. 1, the device 1 for assisting with improvement in the ADLs is illustrated in a standalone state deployed singly to a training facility, but is not limited thereto. In other words, the device 1 for assisting with improvement in the ADLs may also be deployed to each of a plurality of training areas in a training facility. In the case where a plurality of devices 1 for assisting with improvement in the ADLs are provided, the plurality of devices 1 for assisting with improvement in the ADLs may be connected to a central server over a communication network such as a local area network (LAN), and a storage unit in the server may be shareable as a database. Moreover, the plurality of devices 1 for assisting with improvement in the ADLs each share functions with the server. Note that since it would be easy for a person skilled in the art to understand and implement the above modified configuration, the modified configuration is omitted from the drawings and detailed description herein.

Referring to FIG. 2, the device 1 for assisting with improvement in the ADLs is achievable with an information processing device such as a tablet device, a laptop personal computer, or a desktop personal computer, and includes the following components as a hardware configuration. Namely, the device 1 for assisting with improvement in the ADLs is provided with a central processing unit (CPU) 20 as a processor, random access memory (RAM) 21 as a working memory, and read-only memory (ROM) 22 that stores a boot program for bootup.

The device 1 for assisting with improvement in the ADLs is also provided with a disk as non-volatile flash memory 23 for rewritable storage of an operating system (OS), an application program, and various information (including data).

If necessary, depending on the modes of deployment, usage, and the like, the device 1 for assisting with improvement in the ADLs is further provided with a communication control unit 24 that provides wired or wireless communication, a communication interface unit 25 such as a network interface card (NIC), and an imaging unit 26 including a camera.

The device 1 for assisting with improvement in the ADLs is further provided with components such as a display unit 27 including a display (liquid crystal display (LCD)), a display control unit 28, and an information input/designation unit 29 including a keypad, various function buttons (keys), a pointing unit, a cursor-manipulating unit, and the like.

As one example, to logically implement the functions described above and other functions to be described in detail later in the device 1 for assisting with improvement in the ADLs, a program for assisting with improvement in the activities of daily living is installed as an application program in the flash memory 23. Thereafter, when the device 1 for assisting with improvement in the ADLs is triggered by an instruction from a training supervisor or by being powered on, the processor (CPU) 20 loads the program for assisting with improvement in the activities of daily living into the RAM 21 and executes the program.

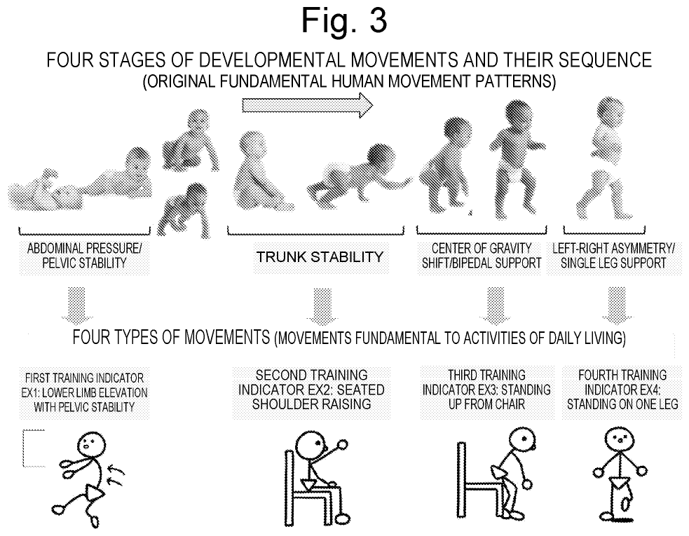

Described in detail with reference to FIGS. 1, 2, and related drawings, a training supervisor (including a licensed therapist and therapist's assistant) launches the program for assisting with improvement in the activities of daily living on the device 1 for assisting with improvement in the ADLs, and then, on a pre-setup screen (not illustrated) displayed on the display unit 27, defines the at least four stages of human developmental movements and their sequence in association with a first training indicator EX1, a second training indicator EX2, a third training indicator EX3, and a fourth training indicator EX4 of the four types of movements (see FIG. 3).

As exemplified in FIG. 3, the four stages of developmental movements and their sequence correspond to original fundamental human movement patterns, and include abdominal pressure and pelvic stability, trunk stability, center of gravity shift and bipedal support, and left-right asymmetry and single leg support. Additionally, the four types of movements associated with the four stages of developmental movements and their sequence are movements fundamental to the activities of daily living, and include straight leg raising (also simply referred to as A-SLR), seated shoulder raising, standing up from a chair, and standing on one leg.

The definitions, including the related images, of the four stages of development movements and their sequence and the four types of movements exemplified in FIG. 3 are stored in the flash memory 23 that forms a storage unit in the device 1 for assisting with improvement in the ADLs in response to an operation for designating an OK button on the pre-setup screen.

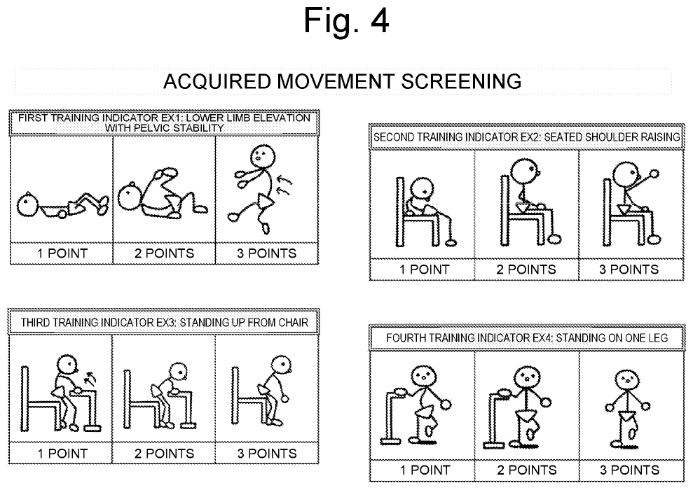

Next, as exemplified in FIG. 4, on the pre-setup screen, the training supervisor defines the first training indicator EX1, the second training indicator EX2, the third training indicator EX3, and the fourth training indicator EX4 of the four types of movements associated with the four stages of developmental movements and their sequence such that each includes three sub-movements categorized into different difficulties corresponding to a plurality of different scores.

Here, the three sub-movements categorized into different difficulties include reference images for clearly presenting and explaining the training content to the training subject. The reference images may be representational images or real images. The plurality of different scores related to each of the reference images are reference scores (points: 1, 2, 3) for evaluating a training subject's level of self-reliance.

The definitions of the four types of movements exemplified in FIG. 4 are stored with respect to each training indicator in the flash memory 23 that forms a storage unit in the device 1 for assisting with improvement in the ADLs in response to an operation for designating an OK button on the pre-setup screen.

The definitions exemplified in FIG. 4 and the definitions exemplified in FIG. 3 are stored in the flash memory 23 so as to be linked with each other, and therefore can be mutually referenced by an operation (for example, a double-click) for designating one of the first training indicator EX1, the second training indicator EX2, the third training indicator EX3, and the fourth training indicator EX4.

In the device 1 for assisting with improvement in the ADLs, a training subject is screened for acquired movements according to a training program which is based on original fundamental human movement patterns and in which a basic sequence or a modified sequence of the four types of movements corresponding to the movement patterns is adopted.

Here, the basic sequence of the four types of movements is the sequence of the first training indicator EX1, the second training indicator EX2, the third training indicator EX3, and the fourth training indicator EX4. Also, a modified sequence of the four types of movements is a sequence omitting at least one subordinate indicator of the first training indicator EX1, the second training indicator EX2, the third training indicator EX3, and the fourth training indicator EX4.

On a training execution screen (not illustrated) displayed on the display unit 27, when triggered by the training supervisor specifying a training subject by name or by identification information (an ID) and performing an operation for designating an initial training start button, all of the definitions of the four types of movements exemplified in FIG. 4 are read out from the flash memory 23 and processed for display on the display unit 27 as a training program (first training program) adopting the basic sequence of the four types of movements.

The training supervisor screens the training subject for acquired movements on the basis of the basic sequence of the four types of movements according to the displayed first training program, and determines an achievement score (points: 0, 1, 2, 3) for the training subject.

The determined achievement score is processed for display on the display unit 27 for each of the first training indicator EX1, the second training indicator EX2, the third training indicator EX3, and the fourth training indicator EX4, and is also stored in a storage area (record) pertaining to the training subject in a training result table created in the flash memory 23.

Although omitted from illustration in the drawings, the training result table has a storage area for each training subject, the storage area at least including the name, identification information, and attributes (including sex, gender, address, and the like) of the training subject, information related to initial training (including a training date, the type of training program, the achievement score, the Barthel Index, and the like), and information related to ongoing training (including a training date, the type of training program, the achievement score, the Barthel Index, and the like).

As described in detail later, in one example, an achievement score for the training subject is determined, in relation to three sub-movements categorized into different difficulties, on the basis of a positive or negative determination regarding an acquired movement and obtained via a selective designation operation by the training supervisor.

Note that, as described in detail in a modification, it is also possible to adopt a processing method in which an achievement score for the training subject is determined, in relation to three sub-movements categorized into different difficulties, on the basis of an automatic positive or negative determination regarding an acquired movement and obtained via image capture.

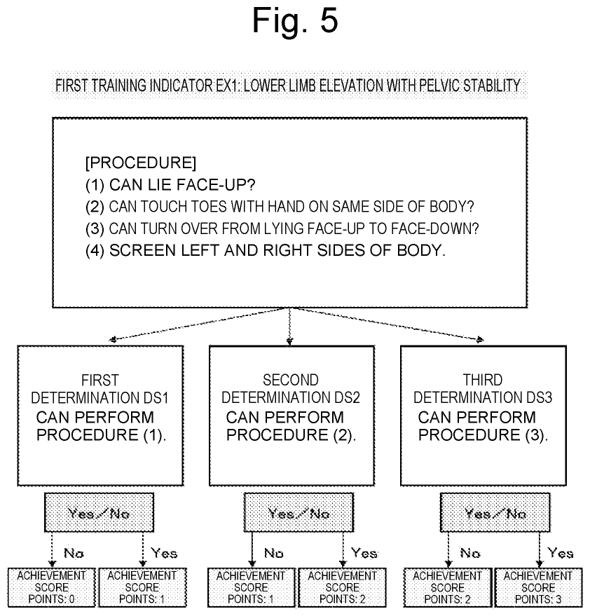

Specific examples of the first training indicator EX1, the second training indicator EX2, the third training indicator EX3, and the fourth training indicator EX4 of the four types of movements used to screen a training subject for acquired movements are predefined as indicated below and stored in the flash memory 23 (see FIGS. 5, 6, 7, and 8). Note that the sub-movements of different difficulties included in the first training indicator EX1, the second training indicator EX2, the third training indicator EX3, and the fourth training indicator EX4 of the four types of movements may be changed within a scope that does not deviate from the purpose of each screening.

First training indicator EX1: straight leg raising (A-SLR) (see FIG. 5)

### [Purpose]

This screening is for evaluating pelvic stabilization ability and the mobility of the hip joint, the knee joint, and the ankle joint.

### [Procedure]

(1) Can lie face-up?
(2) Can touch toes with hand on same side of body?
(3) Can turn over from lying face-up to face-down?
(4) Screen left and right sides of body.

### [Reference Scores]

3 Points: Can perform procedure (3).
2 Points: Can perform procedure (2).
1 Point: Can perform procedure (1).

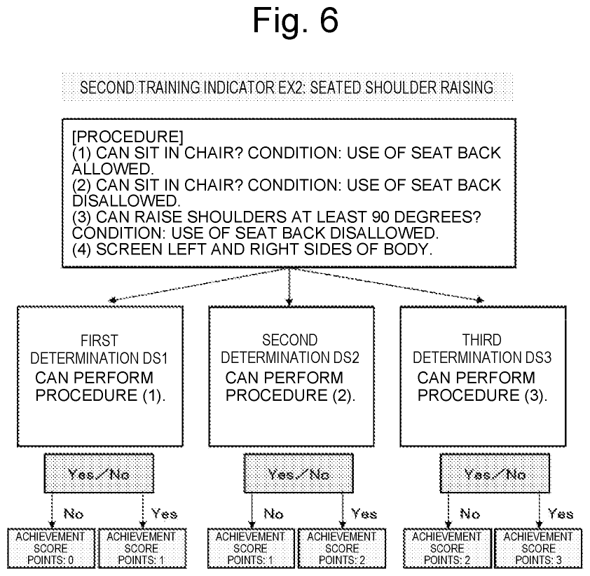

Second training indicator EX2: seated shoulder raising (see FIG. 6)

### [Purpose]

This screening is for evaluating trunk stabilization ability (maintaining a sitting position) and the mobility of the shoulder joint.

### [Procedure]

(1) Can sit in chair? Condition: use of seat back allowed.
(2) Can sit in chair? Condition: use of seat back disallowed.
(3) Can raise shoulders at least 90 degrees? Condition: use of seat back disallowed.
(4) Screen left and right sides of body.

### [Reference Scores]

3 Points: Can perform procedure (3).
2 Points: Can perform procedure (2).
1 Point: Can perform procedure (1).

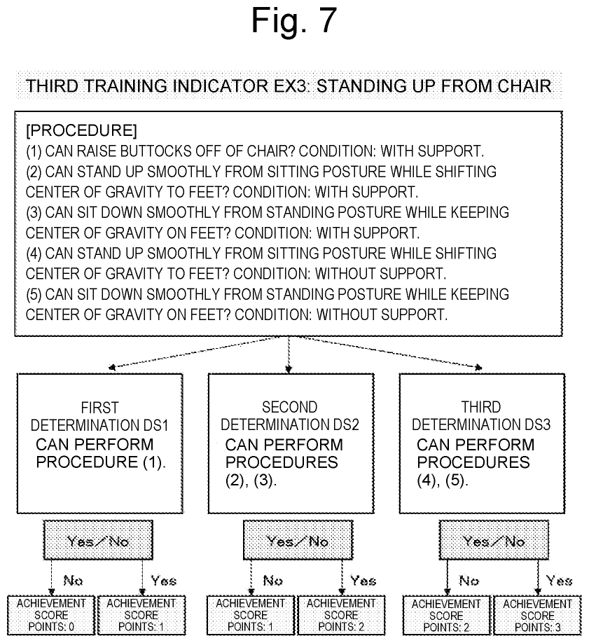

Third training indicator EX3: standing up from chair (see FIG. 7)

### [Purpose]

This screening is for evaluating whether the change of position from sitting to standing and from standing to sitting are performed safely by bending the hip joint while controlling the trunk to shift one's center of gravity smoothly from the buttocks to the feet and from the feet to the buttocks.

### [Procedure]

(1) Can raise buttocks off of chair?
Condition: with support.
(2) Can stand up smoothly from sitting posture while shifting center of gravity to feet?
Condition: with support.
(3) Can sit down smoothly from standing posture while keeping center of gravity on feet?
Condition: with support.
(4) Can stand up smoothly from sitting posture while shifting center of gravity to feet?
Condition: without support.
(5) Can sit down smoothly from standing posture while keeping center of gravity on feet?
Condition: without support.

### [Reference Scores]

3 Points: Can perform procedures (4), (5).
2 Points: Can perform procedures (2), (3).
1 Point: Can perform procedure (1).

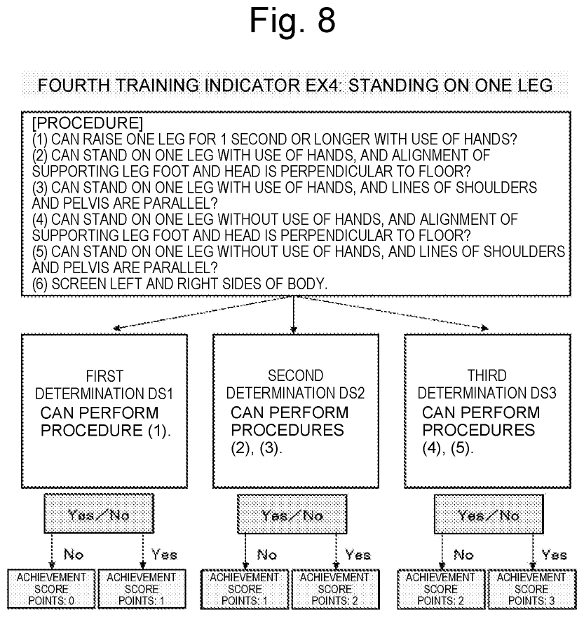

Fourth training indicator EX4: standing on one leg (see FIG. 8)

### [Purpose]

This screening is for evaluating whether static stability can be maintained for each body part in a static state with one leg raised and eyes open, using the foot of the supporting leg as a support surface.

### [Procedure]

(1) Can raise one leg for 1 second or longer with use of hands?
(2) Can stand on one leg with use of hands, and alignment of supporting leg foot and head is perpendicular to floor?
(3) Can stand on one leg with use of hands, and lines of shoulders and pelvis are parallel?
(4) Can stand on one leg without use of hands, and alignment of supporting leg foot and head is perpendicular to floor?
(5) Can stand on one leg without use of hands, and lines of shoulders and pelvis are parallel?
(6) Screen left and right sides of body.

### [Reference Scores]

3 Points: Can perform procedures (4), (5).
2 Points: Can perform procedures (2), (3).
1 Point: Can perform procedure (1).

The definitions of the training indicators described above which are stored in the flash memory 23 (see FIGS. 5, 6, 7, and 8) are associated so as to be linked with the first training indicator EX1, the second training indicator EX2, the third training indicator EX3, and the fourth training indicator EX4 of the four types of movements exemplified in FIG. 4. Consequently, by performing an operation (for example, a single-click) for designating one of the first training indicator EX1, the second training indicator EX2, the third training indicator EX3, and the fourth training indicator EX4 of the four types of movements illustrated in FIG. 4 and displayed on the training execution screen, the training supervisor can refer to and present the definition of the corresponding training indicator to the training subject and screen for the acquired movement.

As described above, when the training supervisor screens the training subject for acquired movements on the basis of the basic sequence of the four types of movements according to the first training program, in the device 1 for assisting with improvement in the ADLs, an achievement score (points: 0, 1, 2, 3) for the training subject is determined automatically in relation to three sub-movements categorized into different difficulties and on the basis of a positive or negative determination regarding acquired movements obtained via a selective designation operation by the training supervisor.

The determination method will be described in detail later, but as an example, in relation to the straight leg raising (A-SLR) of the first training indicator EX1 in the basic sequence of the four types of movements, when the training subject is screened for acquired movements according to the three sub-movements categorized into different difficulties, the training supervisor performs an operation for selectively designating a positive determination (Yes) or a negative determination (No) regarding the acquired movements on the basis of the displaying of a processing procedure exemplified in FIG. 5.

In the device 1 for assisting with improvement in the ADLs, by totaling the positive determinations (Yes) and the negative determinations (No) regarding acquired movements, an achievement score for the training subject can be determined automatically. For example, when a first determination DS1, a second determination DS2, and a third determination DS3 in the processing procedure for the straight leg raising screening exemplified in FIG. 5 are all positive determinations (Yes), the achievement score for the training subject is determined to be 3 points. Note that when the first determination DS1 is a negative determination (No), the achievement score for the training subject is determined to be 0 points. The determined achievement score is stored in the storage area (record) pertaining to the training subject in the training result table.

Next, on the training execution screen displayed on the display unit 27, if the training supervisor specifies a training subject by name or by identification information (an ID) and performs an operation for designating an ongoing training start button, in the device 1 for assisting with improvement in the ADLs, a training program (second training program) adopting the basic sequence or a modified sequence of the four types of movements is created (generated) according to the previous achievement score for each training indicator. Thereafter, the generated second training program is processed for display on the display unit 27 to screen for acquired movements.

Here, the generation of the second training program adopting the basic sequence of the four types of movements corresponds to all of the definitions of the four types of movements exemplified in FIG. 4 being read out from the flash memory 23 and processed for display on the display unit 27.

When the achievement score is low for all of the first training indicator EX1, the second training indicator EX2, the third training indicator EX3, and the fourth training indicator EX4 in the training result table regarding the four types of movements (for example, all scores of 0 or 1), the second training program is generated in the basic sequence.

Thereafter, the method relating to screening the training subject for acquired movements on the basis of the basic sequence of the four types of movements according to the second training program and determining the achievement score (points: 0, 1, 2, 3) for the training subject is the same as the process based on the basic sequence of the four types of movements according to the first training program described above.

Also, the generation of the second training program adopting a modified sequence of the four types of movements corresponds to the definitions of the four types of movements exemplified in FIG. 4 being read out from the flash memory 23 and processed for display on the display unit 27 in a sequence omitting at least one subordinate indicator of the first training indicator EX1, the second training indicator EX2, the third training indicator EX3, and the fourth training indicator EX4.

However, when generating the second training program adopting a modified sequence of the four types of movements, the following rules are applied. When the achievement score is the same in adjacent sequence of first training indicator EX1, the second training indicator EX2, the third training indicator EX3, and the fourth training indicator EX4 in the training result table regarding the four types of movements, the second training program is generated in a modified sequence adopting the lower-order training indicator.

In other words, when the achievement scores for the first training indicator EX1, the second training indicator EX2, the third training indicator EX3, and the fourth training indicator EX4 in the training result table are 3, 2, 2, 2 points, for example, by determining that the second training indicator EX2 and the third training indicator EX3 have the same score of 2 points adjacent in sequence, the second training program is generated in a modified sequence (second training indicator EX2, third training indicator EX3, fourth training indicator EX4) adopting the lower-order second training indicator EX2.

Also, when the first training indicator EX1, the second training indicator EX2, the third training indicator EX3, and the fourth training indicator EX4 in the training result table regarding the four types of movements indicate different evaluations on the left and right sides of the training subject's body, the second training program is generated in a modified sequence adopting the training indicator with the lower achievement score.

In other words, when the achievement score for the second training indicator EX2 in the training result table is 3 points for the right side of the body and 2 points for the left side of the body, for example, by determining that there is a difference between the left and right sides of the body, the second training program is generated in a modified sequence adopting the second training indicator EX2 for the lower achievement score (2 points for the left side of the body) in consideration of the other training indicators.

When the training supervisor screens the training subject for acquired movements on the basis of a modified sequence of the four types of movements according to the second training program, in the device 1 for assisting with improvement in the ADLs, an achievement score (points: 0, 1, 2, 3) for the training subject is determined automatically in relation to three sub-movements categorized into different difficulties and on the basis of a positive or negative determination regarding acquired movements obtained via a selective designation operation by the training supervisor.

Regarding the determination method, as described in the basic sequence of the four types of movements, when the training subject is screened for acquired movements according to the three sub-movements categorized into different difficulties, the training supervisor performs an operation for selectively designating a positive determination (Yes) or a negative determination (No) regarding the acquired movements on the basis of the displaying of a processing procedure exemplified in FIG. 5.

In the device 1 for assisting with improvement in the ADLs, by totaling the positive determinations (Yes) and the negative determinations (No) regarding acquired movements, an achievement score for the training subject can be determined automatically. The determined achievement score is stored in the storage area (record) pertaining to the training subject in the training result table.

In the device 1 for assisting with improvement in the ADLs described above, the goal is not to reach a total of 12 points in the achievement score for the training subject, but to reach 2 points or more for all four types of movements.

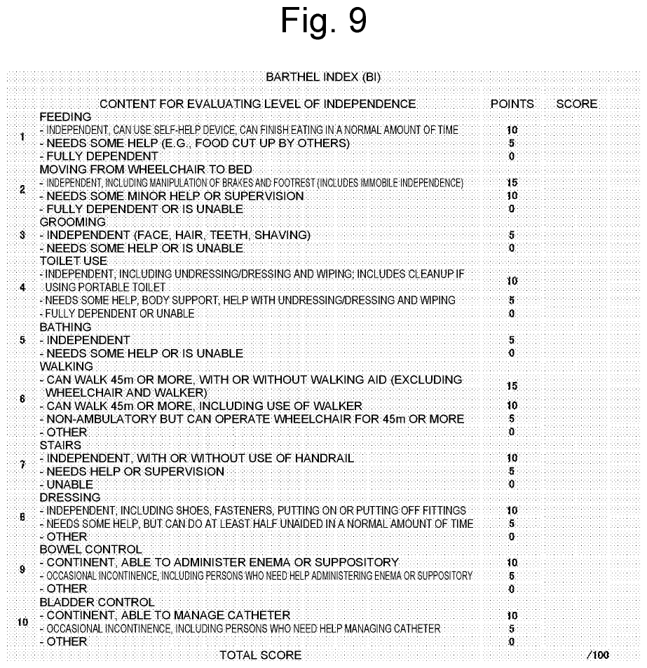

By the way, the Barthel Index (BI) is an index typically used to evaluate the level of independence on multiple levels, such as independent, partially dependent, and fully dependent, with respect to 10 items of independence evaluation content, namely “feeding”, “moving from wheelchair to bed”, “grooming”, “toilet use”, “bathing”, “walking”, “stairs”, “dressing”, “bowel control”, and “bladder control”, as illustrated in FIG. 9.

In the Barthel Index (BI), points are set according to the level of independence, and the case of being fully independent is 100 points. As a rough estimate, it is inferred that a total score of 40 points or less necessitates help with almost all items, while 60 points or less mainly necessitates help with getting up and moving around.

In the device 1 for assisting with improvement in the ADLs described above, the ultimate goal is to improve the scores (ADL values) on the Barthel Index (BI) which is an index indicating a training subject's level of independence in the activities of daily living. Consequently, the training subject's level of self-reliance is evaluated with consideration for both the score on the Barthel Index (BI) obtained from answers to questions asked of the training subject at the beginning of initial training and the beginning of ongoing training, and the achievement score for the training subject in the four types of movements.

Additionally, regarding the score on the Barthel Index (BI) obtained from answers to questions asked of the training subject at the beginning of initial training and the beginning of ongoing training, if there are different scores for the left and right sides of the training subject's body, the higher score is adopted.

[Process for Assisting with Improvement in the Activities of Daily Living]

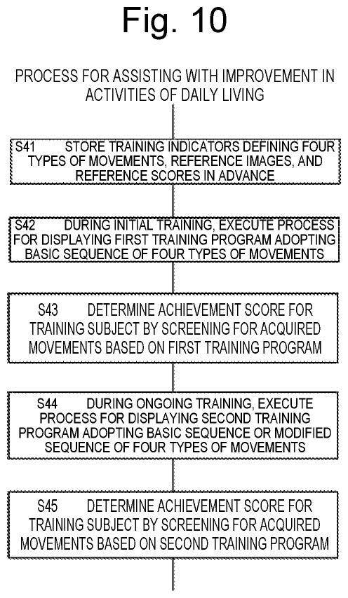

Next, operations in the device 1 for assisting with improvement in the ADLs described above will be described with reference to FIGS. 1, 2, and related drawings. FIG. 10 illustrates one example of a sequence of a process for assisting with improvement in the activities of daily living in the device 1 for assisting with improvement in the ADLs described above.

The device 1 for assisting with improvement in the ADLs is triggered by being powered on by the training supervisor, a processing program is launched, and the processor (CPU) 20 executes the process for assisting with improvement in the activities of daily living described below in cooperation with the training supervisor.

[Process S41 (see FIG. 10)] Training indicators defining the four types of movements, reference images, and reference scores are stored in advance.

In other words, training indicators defining the four types of movements associated with at least four stages of human developmental movements and their sequence, reference images of the four types of movements, and reference scores for evaluating a training subject's level of self-reliance are stored in advance for each training indicator in the storage unit 23.

[Process S42] During initial training, the first training program adopting the basic sequence of the four types of movements is processed for display.

In other words, the start of initial training for the training subject triggers the processing of the first training program adopting the basic sequence of the four types of movements for display on the display unit 27.

[Process S43] By screening for acquired movements based on the first training program, an achievement score for the training subject is determined.

In other words, by screening for acquired movements based on the first training program, an achievement score for the training subject is determined, processed for display on the display unit 27, and stored for each training indicator in the storage unit 23.

[Process S44] During ongoing training, the second training program adopting the basic sequence or a modified sequence of the four types of movements is processed for display.

In other words, the start of ongoing training for the training subject triggers the processing of the second training program adopting the basic sequence or a modified sequence of the four types of movements according to the achievement score for each training indicator for display on the display unit 27.

[Process S45] By screening for acquired movements based on the second training program, an achievement score for the training subject is determined.

In other words, by screening for acquired movements based on the second training program, an achievement score for the training subject is determined, processed for display on the display unit 27, and stored for each training indicator in the storage unit 23.

### Features and Effects of Embodiment

In the device 1 for assisting with improvement in the ADLs according to the embodiment described above, training indicators defining the four types of movements associated with at least four stages of human developmental movements and their sequence, reference images of the four types of movements, and reference scores for evaluating a training subject's level of self-reliance are stored in advance for each training indicator in the storage unit 23.

Additionally, in the device 1 for assisting with improvement in the ADLs, the start of initial training for the training subject triggers the processing of the first training program adopting the basic sequence of the four types of movements for display on the display unit 27, and by screening for acquired movements based on the first training program, an achievement score for the training subject is determined, processed for display on the display unit 27, and stored for each training indicator in the storage unit 23.

Also, in the device 1 for assisting with improvement in the ADLs, the start of ongoing training for the training subject triggers the processing of the second training program adopting the basic sequence or a modified sequence of the four types of movements according to the achievement score for each training indicator for display on the display unit 27, and by screening for acquired movements based on the second training program, an achievement score for the training subject is determined, processed for display on the display unit 27, and stored for each training indicator in the storage unit 23.

With this arrangement, it is possible to rapidly screen for movements that the training subject needs to acquire at the present time and prescribe a proper training regimen for the movements that need to be acquired, thereby leading to improvements in the ADLs for the training subject.

Additionally, the device 1 for assisting with improvement in the ADLs according to the embodiment described above has at least the following effects.
(1) An entry point for performing training (exercise) can be discovered for each training subject aiming to improve their ADLs. Furthermore, by setting a movement that is almost but not quite achievable as an entry point and modifying the parameters for performing the movement, a training program can be created on a level that is achievable with effort.
(2) Movements and exercises that are desirable for the training subject to acquire can be prescribed as a set. Furthermore, an understanding of the meaning of the exercises is fostered, thereby leading to effective exercise performance and increased motivation.
(3) Since movements and exercises that are desirable to acquire are clarified, groups can be formed on the basis thereof, making training manageable even with few training supervisors.

### Modification of Embodiment

In the device 1 for assisting with improvement in the ADLs according to the embodiment described above, the modification described below can be adopted.

The device 1 for assisting with improvement in the ADLs according to the embodiment described above illustrates an example (see FIGS. 4 and 5-8) in which an achievement score (points: 0, 1, 2, 3) for the training subject is determined, in relation to three sub-movements categorized into different difficulties corresponding to a plurality of different scores, on the basis of a positive (Yes) or negative (No) determination regarding an acquired movement and obtained via a selective designation operation by the training supervisor.

In the device 1 for assisting with improvement in the

ADLs according to the modification, it is also possible to implement a processing method in which the achievement score for the training subject is determined, in relation to three sub-movements categorized into different difficulties corresponding to a plurality of different scores, on the basis of an automatic positive or negative determination regarding an acquired movement and obtained by image analysis through image capture by the imaging unit 26 including a camera.

### Other Embodiments

The processes in the embodiment and modification described above may be provided as a computer-executable program, may be provided via a non-transitory computer-readable recording medium, such as a CD-ROM or a flexible disk, and may also be provided via a communication channel.

In addition, any number or all of the processes in the embodiment and modification described above can be selectively combined and carried out.

### DESCRIPTION OF THE REFERENCE NUMERALS

1 device for assisting with improvement in the activities of daily living (ADLs)
EX1 first training indicator (straight leg raising)
EX2 second training indicator (seated shoulder raising)
EX3 third training indicator (standing up from chair)
EX4 fourth training indicator (standing on one leg)

1. A device for assisting with improvement in activities of daily living that provides a training program for assisting with improvement in the activities of daily living for a training subject, the device comprising a processor configured to:
store in advance training indicators defining four types of movements associated with at least four stages of human developmental movements and their sequence, reference images of the four types of movements, and reference scores for evaluating a training subject's level of self-reliance for each training indicator in a storage unit;
process a first training program adopting a basic sequence of the four types of movements for display on a display unit, upon being triggered by the start of initial training for the training subj ect;
screen for acquired movements based on the first training program to determine an achievement score for the training subject, and for each training indicator, process the achievement score for display on the display unit and store the achievement score in the storage unit;
process a second training program adopting the basic sequence or a modified sequence of the four types of movements according to the achievement score for each training indicator for display on the display unit, upon being triggered by the start of ongoing training for the training subject; and
screen for acquired movements based on the second training program to determine an achievement score for the training subject, and for each training indicator, process the achievement score for display on the display unit and store the achievement score in the storage unit.

2. The device for assisting with improvement in the activities of daily living according to claim 1,
wherein the four stages of developmental movements and their sequence correspond to original fundamental human movement patterns, and include abdominal pressure and pelvic stability, trunk stability, center of gravity shift and bipedal support, and left-right asymmetry and single leg support.

3. The device for assisting with improvement in the activities of daily living according to claim 2,
wherein the four types of movements are movements fundamental to the activities of daily living, and include straight leg raising, seated shoulder raising, standing up from a chair, and standing on one leg as first, second, third, and fourth training indicators, respectively.

4. The device for assisting with improvement in the activities of daily living according to claim 3,
wherein the basic sequence of the four types of movements is the sequence of the first training indicator, the second training indicator, the third training indicator, and the fourth training indicator.

5. The device for assisting with improvement in the activities of daily living according to claim 3,
wherein the modified sequence of the four types of movements is a sequence omitting at least one subordinate indicator of the first training indicator, the second training indicator, the third training indicator, and the fourth training indicator.

6. The device for assisting with improvement in the activities of daily living according to claim 3,
wherein the first training indicator, the second training indicator, the third training indicator, and the fourth training indicator of the four types of movements each include three sub-movements categorized into different difficulties corresponding to a plurality of different scores.

7. The device for assisting with improvement in the activities of daily living according to claim 3,
wherein the first training indicator, the second training indicator, the third training indicator, and the fourth training indicator of the four types of movements each include three sub-movements categorized into different difficulties corresponding to a plurality of different scores, and
the processor is configured to determine the achievement score for the training subject on a basis of a positive or negative determination regarding an acquired movement to be obtained via a selective designation with respect to the three sub-movements categorized into different difficulties.

8. The device for assisting with improvement in the activities of daily living according to claim 3,
wherein the first training indicator, the second training indicator, the third training indicator, and the fourth training indicator of the four types of movements each include three sub-movements categorized into different difficulties corresponding to a plurality of different scores, and
the processor is configured to determine the achievement score for the training subject on a basis of a positive or negative determination regarding an acquired movement to be obtained by image analysis via image capture of the three sub-movements categorized into different difficulties.

9. The device for assisting with improvement in the activities of daily living according to claim 3,
wherein when the achievement score is low for all of the first training indicator, the second training indicator, the third training indicator, and the fourth training indicator of the four types of movements, the processor is configured to generate the second training program in the basic sequence.

10. The device for assisting with improvement in the activities of daily living according to claim 3,
wherein when the achievement score is the same in adjacent sequence of the first training indicator, the second training indicator, the third training indicator, and the fourth training indicator of the four types of movements, the processor is configured to generate the second training program in a modified sequence adopting the lower-order training indicator.

11. The device for assisting with improvement in the activities of daily living according to claim 3,
wherein when the first training indicator, the second training indicator, the third training indicator, and the fourth training indicator of the four types of movements indicate different evaluations on the left and right sides of the training subject's body, the processor is configured to generate the second training program in a modified sequence adopting the training indicator with the lower achievement score.

12. The device for assisting with improvement in the activities of daily living according to claim 1,
wherein the processor is configured to evaluate the training subject's level of self-reliance with consideration for both a score on the Barthel Index which is an index indicating the training subject's level of independence in the activities of daily living, and the achievement score for the training subject in the four types of movements.

13. The device for assisting with improvement in the activities of daily living according to claim 12,
wherein when the index indicating the level of independence in the activities of daily living indicates different evaluations on the left and right sides of the training subject's body, the processor is configured to adopt the higher score.

14. A method for assisting with improvement in activities of daily living that provides a training program for assisting with improvement in the activities of daily living for a training subject, the method being executed by a processor and comprising:
storing in advance training indicators defining four types of movements associated with at least four stages of human developmental movements and their sequence, reference images of the four types of movements, and reference scores for evaluating a training subject's level of self-reliance for each training indicator in a storage unit;
processing a first training program adopting a basic sequence of the four types of movements for display on a display unit, upon being triggered by the start of initial training for the training subject;
screening for acquired movements based on the first training program to determine an achievement score for the training subject, and for each training indicator, processing the achievement score for display on the display unit and storing the achievement score in the storage unit;
processing a second training program adopting the basic sequence or a modified sequence of the four types of movements according to the achievement score for each training indicator for display on the display unit, upon being triggered by the start of ongoing training for the training subject; and
screening for acquired movements based on the second training program to determine an achievement score for the training subject, and for each training indicator, processing the achievement score for display on the display unit and storing the achievement score in the storage unit.

15. A non-transitory computer readable medium recorded with a program for assisting with improvement in activities of daily living that provides a training program for assisting with improvement in the activities of daily living for a training subject, the program causing a processor to execute a process comprising:
storing in advance training indicators defining four types of movements associated with at least four stages of human developmental movements and their sequence, reference images of the four types of movements, and reference scores for evaluating a training subject's level of self-reliance for each training indicator in a storage unit;
processing a first training program adopting a basic sequence of the four types of movements for display on a display unit, upon being triggered by the start of initial training for the training subject;
screening for acquired movements based on the first training program to determine an achievement score for the training subject, and for each training indicator, processing the achievement score for display on the display unit and storing the achievement score in the storage unit;
processing a second training program adopting the basic sequence or a modified sequence of the four types of movements according to the achievement score for each training indicator for display on the display unit, upon being triggered by the start of ongoing training for the training subject; and
screening for acquired movements based on the second training program to determine an achievement score for the training subject, and for each training indicator, processing the achievement score for display on the display unit and storing the achievement score in the storage unit.
